(19) United States
(12) Patent Application Publication
(10) Pub. No.: US 2025/0101459 A1
(43) Pub. Date: Mar. 27, 2025

(54) METHODS FOR CREATING LINKAGE OF MULTIPLE VIRAL VECTORS FOR INTRACELLULAR DELIVERY

(71) Applicant: Battelle Memorial Institute, Columbus, OH (US)

(72) Inventors: David GLASBRENNER, Hilliard, OH (US); David ALBERTSON, Dublin, OH (US)

(73) Assignee: BATTELLE MEMORIAL INSTITUTE, Columbus, OH (US)

(21) Appl. No.: 18/892,177

(22) Filed: Sep. 20, 2024

Related U.S. Application Data
(60) Provisional application No. 63/584,196, filed on Sep. 21, 2023.

(51) Int. Cl. C12N 15/86

(52) U.S. Cl. CPC C12N 15/86; C12N 2750/14143; C12N 2750/14151

(57) ABSTRACT

Methods and structures are disclosed for creating a physical linkage between two or more viral particles, which can covalently link the viral particles together. The methods and structures described herein are designed for purposes of improving efficiency and effectiveness of vector delivery into cells and tissues for purposes of gene therapy. Methods for linking two or more viral vectors comprise functionalizing a first vector with a first surface moiety and functionalizing a second vector with a second surface moiety. Thereafter, the first functionalized vector and second functionalized vector are combined so that the two surface moieties can react. During this reaction, the first surface moiety and the second surface moiety form a covalent linkage, thereby resulting in the physical linkage of both vectors to each other.

### CROSS-REFERENCE TO RELATED APPLICATIONS

This is a U.S. Non-Provisional Patent Application which claims benefit and priority to U.S. Provisional Patent Application Ser. No. 63/584,196, filed on Sep. 21, 2023, titled “METHODS FOR CREATING LINKAGE OF MULTIPLE VIRAL VECTORS FOR INTRACELLULAR DELIVERY”, the content and disclosure of which is incorporated herein in its entirety.

### TECHNICAL FIELD

The present disclosure relates to methods of creating a physical linkage between multiple viral particles. Methods and structures are provided for linking viral particles, and particularly Adeno-Associated Virus (AAV) particles, for purposes of intracellular delivery for gene therapies.

### BACKGROUND OF INVENTION

Gene therapy functions by introducing or administering genetic materials into a subject with the aim of altering gene or protein expression, and potentially providing a curative treatment for many diseases that currently have no cure. The research conducted in the last few decades has led to a prevalence of specific viral strains used as delivery vehicles for the targeted gene structures. In recent years, adeno-associated viruses (AAVs) have shown to be effective delivery vehicles, or vectors, to deliver genes of interest into a broad range of cell types in gene therapy. Hereditary diseases are particularly attractive targets; they are caused by gene mutations, resulting in deficiency or malfunction of proteins required for cellular functions. To treat such hereditary diseases at their source, gene therapy can theoretically correct gene mutations in three ways-(1) by replacing the defective gene with a normal functioning copy, (2) by silencing the mutated version of the gene, and (3) by adding or overexpressing a therapeutic gene or synthetic construct.

Adeno-associated virus belongs to the parvovirus family and is dependent on co-infection with other viruses, mainly adenoviruses, in order to replicate. Initially distinguished serologically, molecular cloning of AAV genes has identified hundreds of unique AAV strains in numerous species. The structure of a typical AAV particle is comprised of a protein shell surrounding and protecting a small, single-stranded DNA genome of approximately 4.8 kilobases (kb), although this is not the form used for gene therapies. Instead, recombinant AAV is utilized. Recombinant AAV (rAAV), which lacks viral DNA, is essentially a protein-based nanoparticle that can be engineered to traverse the cell membrane and deliver its DNA cargo into the nucleus of the cell. In practical terms, rAAVs are composed of the same capsid sequence and structure as found in wild-type AAVs. However, rAAVs encapsulate genomes that are devoid of all AAV protein-coding sequences and instead have therapeutic gene expression cassettes designed in their place. Because rAAVs typically only accommodate genomes that are under 5.0 kb, the genetic structure or “cargo” its transporting must be carefully designed to consider not only the therapeutic transgene sequence but also the inclusion of regulatory elements necessary for gene expression (such as for example, the promoter).

There are various hurdles researchers face when designing an effective and safe gene therapy delivery method for a specific disease. Current methods of delivering more than one AAV to a single cell require a substantial amount of AAV vectors administered to a subject, to improve likelihood that two separate AAVs enter the same cell. Even if an AAV particle or vector can successfully be endocytosed by the cell, it must then travel towards the nucleus in order to successfully deliver its genetic cargo. Studies estimate only about 30% of AAV particles will successfully reach the nucleus of the cell. Therefore, consistent and reproducible delivery of more than one gene to the same cell requires very high doses be administered, as the odds of two separate viral particles encountering the identical cell can be problematic and unreliable. Due to these significant hurdles, multi-gene delivery is often not achievable in vivo.

This is also true for delivery of sub-fragments of a larger DNA target, such as a large transgene. AAVs are limited in accommodating large transgenes due to their small packaging size (about 5.0 kb). Dystrophin for example, a vital protein involved in muscle fiber strengthening, mutations of which cause Duchenne muscular dystrophy, is encoded by a gene that is about 11.5 kb. This exceeds the packaging limit of a single AAV particle. Researchers have attempted to solve the problem of delivering oversized transgenes in rAAVs by co-administering two AAV vectors that carry separate halves of the gene encoding this protein. This approach also faces similar hurdles in that both AAV vectors carrying the separate fragments of the genes have to enter the same cell and thereafter the nucleus. Increasing the likelihood that both vectors enter a single cell requires administering large doses of AAV vectors to a patient; this can result in severe complications, largely due to the body's immunogenic response.

In light of the foregoing disadvantages, there remains a need for the development of methods that ensure delivery of multiple AAV vectors into a single cell. There also remains a need for improved methods of delivery of multiple genes or different gene fragments into a cell. There is a further need for development of methods that can significantly reduce the dose of AAVs administered to a subject, so as to mitigate large dose related complications due to immunogenic responses.

### SUMMARY

Provided herein are methods and structures for creating a physical linkage between two or more viral particles, which can covalently link the viral particles together. The methods and structures described herein are designed for purposes of improving efficiency and effectiveness of vector delivery into cells and tissues for purposes of gene therapy. By creating a physical linkage between multiple AAV vectors, these methods ensure that if one AVV vector is able to enter the cell, then a second, physically linked AAV vector will also enter that same cell, thusly greatly increasing the rate of successfully delivering multiple AAV vectors into a single cell. In return, using this delivery method can reduce the amount of the viral payload which is administered to a subject, thereby reducing the immunogenic response associated with administration of large dose AAV therapies.

In one embodiment, a method is disclosed for linking together two or more adeno-associated virus (AAV) vectors or particles. The method comprises the steps of functionalizing a first AAV vector with a first surface moiety and functionalizing a second AAV vector with a second surface moiety. Thereafter, the first functionalized AAV vector and second functionalized AAV vector are combined so that the two surface moieties can react. During this reaction, the first surface moiety and the second surface moiety react to form a covalent linkage, thereby resulting in the physical linkage of both AAV vectors to each other.

In some embodiments, the reaction that forms the covalent linkage between two or more AAV particles is biorthogonal. Biorthogonal reactions include a strain-promoted azide—alkyne click cycloaddition (SPAAC) reaction, a copper catalyzed azide-alkyne cycloaddition (CuAAC), a Staudinger ligation reaction, a strain-promoted alkyne—nitrone cycloaddition (SPANC) reaction, an inverse electron demand Diels-Alder (IEEDD) reaction, or a combination thereof.

In some embodiments, the covalent linkage formed between the first surface moiety and the second surface moiety comprises the following Structure 1:

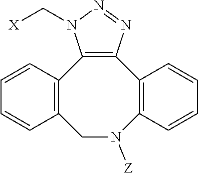

In the resulting Structure 1, X can represent either a linking structure to the first AAV vector or the second AAV vector, and Z can represent either a linking structure to the first AAV vector or a linking structure to the second AAV vector.

The viral particles in the linked systems disclosed herein are designed to be used for purposes of delivering a genetic cargo within cells or tissue. This genetic cargo will broadly be referred to as a nucleic acid construct, for purposes of this disclosure. The nucleic acid construct packaged inside the various viral vectors of the present invention can be any kind of nucleotide sequence that is capable of transduction into cells by a recombinant virus. In further embodiments, the nucleic acid construct is capable of transcription for gene replacement, gene silencing, gene editing, gene addition or a combination thereof.

In certain embodiments, when two viral vectors are linked together, a first vector, for example a first AAV vector encapsulates a first nucleic acid construct and a second AAV vector, encapsulates a second nucleic acid construct. In some embodiments, the first nucleic acid construct and the second nucleic acid construct are the same. In other embodiments, the first nucleic acid construct and the second nucleic acid construct are different. This is the case for example, when different gene fragments are packaged into first AAV and second AAV, for delivery of the different genes to the same cell or tissue, for purposes of multi-gene therapies. This can also be the case where different fragments of the same large gene sequence are packaged into multiple AAV particles, for delivery to the same cell or tissue.

In another embodiment, a method of creating a linkage between three or more viral vectors is disclosed. The method comprises the steps of:
functionalizing a surface of first viral vector with a first reactive group;
functionalizing a surface of a second viral vector with a second reactive group;
functionalizing a surface of a third viral vector with a third reactive group;
providing a heterofunctional linking molecule; and
reacting the first, second and third viral vectors with the heterofunctional linking molecule. The first, second and third reactive moieties attach to the heterofunctional linking molecule to form a covalently linked structure comprising the first, second and third viral vectors.

In a further embodiment, methods comprise use of heterotrifunctional linking molecule, having following structure (structure 2):

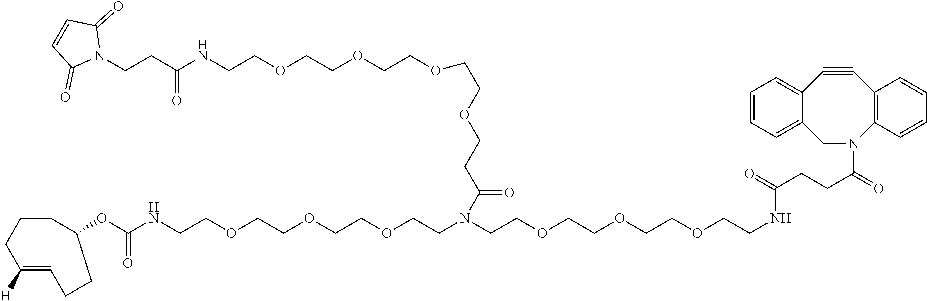

Also disclosed are covalently linked structures and compositions comprising said structures which can be administered to a subject in need of therapeutic gene transfer, gene editing, or gene addition, or a combination thereof.

### Selected Definitions

As used herein, the term “adeno-associated virus” or “AAV” “AAV vector” or “AAV particle” or “AAV virus” or “AAV virion” are used interchangeably and synonymously throughout the disclosure and refer generally to adeno-associated virus of any serotype, including recombinant adeno-associated virus (rAAV).

The term “nucleic acid construct”, as used herein, refers to any structure which comprises a sequence of nucleotides, which already exists within a viral vector and/or which is foreign to and can be packaged into a virus.

The term “heterofunctional molecule”, as used herein refers to a structure that has multiple reactive molecules that are capable of separately binding to a reactive group, or to a target, and are attached together through chemical linking molecule and/or spacer molecules.

The term “heterotrifunctional molecule”, as used herein, refers to a structure that has three reactive molecules or groups that are capable of separately binding to three other reactive groups, and are attached together through chemical linking molecule and/or spacer molecules.

The term “about” is used in conjunction with numeric values to include normal variations in measurements as expected by persons skilled in the art, and is understood to have the same meaning as “approximately” and to cover a typical margin of error, such as ±15%, ±10%, ±5%, ±1%, ±0.5%, or even ±0.1% of the stated value. The term “about” also encompasses amounts that differ due to different equilibrium conditions for a composition resulting from a particular initial composition. Whether or not modified by the term “about,” the claims include equivalents to the quantities.

It should be noted that, as used in this specification and the appended claims, the singular forms “a,” “an,” and “the” include plural referents unless the content clearly dictates otherwise. Thus, for example, reference to a composition containing “a compound” includes having two or more compounds that are either the same or different from each other. It should also be noted that the term “or” is generally employed in its sense including “and/or” unless the content clearly dictates otherwise. As used herein, “and/or” refers to and encompasses any and all possible combinations of one or more of the associated listed items, as well as the lack of combinations when interpreted in the alternative (“or”).

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The term “comprise”, “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 1%, 5%, 10%, 15%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 1%, 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5% or even 1%) detectable activity or amount.

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present disclosure.

As used throughout this description, and in the claims, a list of items joined by the term “at least one of” or “one or more of” can mean any combination of the listed terms. For example, the phrase “at least one of X, Y or Z” can mean X; Y; Z; X and Y; X and Z; Y and Z; or X, Y and Z

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all subcombinations of the various embodiments and elements thereof are also specifically embraced by the present invention and are disclosed herein just as if each and every such subcombination was individually and explicitly disclosed herein

### BRIEF DESCRIPTION OF THE DRAWINGS

FIG. 1 depicts a schematic of a click chemistry reaction for coupling two AAV particles, according to an embodiment of the present invention.

FIG. 2 depicts a structure of a heterotrifunctional linking molecule, in accordance with embodiments of the present invention.

FIG. 3 shows a system where three viral particles are linked together via a heterotrifunctional linkage system, in accordance with embodiments disclosed herein.

FIG. 4 shows a result from DNA gel electrophoresis comparing experimental results of unreacted and reacted/linked AAV particles, via SPAAC click-chemistry reaction, in accordance with examples and embodiments of the present invention.

FIG. 5 depicts a schematic of a methionine chemo-selective ligation reaction for coupling two AAV particles, according to an embodiment of the present invention.

FIG. 6 depicts a schematic of a tyrosine linkage with PTAD derivatives reaction for coupling two AAV particles, according to an embodiment of the present invention

FIG. 7 depicts a schematic of a linkage mechanism method for coupling two AAV particles, according to an embodiment of the present invention.

FIG. 8 depicts a schematic of a linkage mechanism method for coupling three AAV particles, according to an embodiment of the present invention.

FIG. 9 depicts a schematic of a linkage mechanism method for coupling five AAV particles, according to an embodiment of the present invention.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

In one embodiment, a method is disclosed for linking together two or more adeno-associated virus (AAV) vectors or particles. The method comprises the steps of functionalizing a first AAV vector with a first surface moiety and functionalizing a second AAV vector with a second surface moiety. Thereafter, the first functionalized AAV vector and second functionalized AAV vector are combined so that the two surface moieties can react. During this reaction, the first surface moiety and the second surface moiety react to form a covalent linkage, thereby resulting in the physical linkage of both AAV vectors to each other.

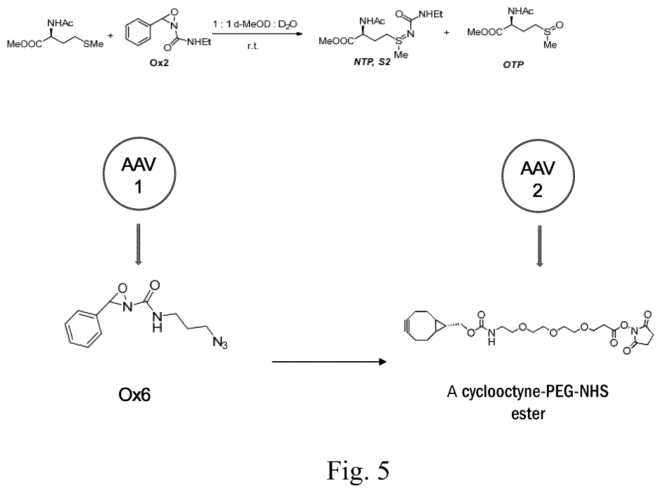

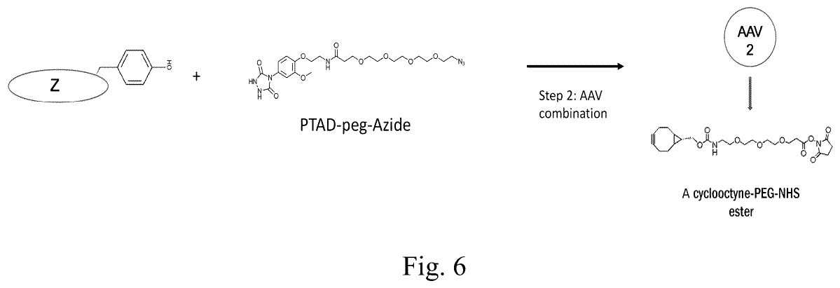

In some embodiments, the reaction that forms the covalent linkage between two or more AAV particles is biorthogonal. Biorthogonal reactions include a strain-promoted azide-alkyne click cycloaddition (SPAAC) reaction, a copper catalyzed azide-alkyne cycloaddition (CuAAC), a Staudinger ligation reaction, a strain-promoted alkyne-nitrone cycloaddition (SPANC) reaction, an inverse electron demand Diels-Alder (IEEDD) reaction. In other embodiments, the reaction that forms the covalent linkage between two or more AAV particles utilizes a Tyrosine linkage via modified PTAD linkers (4-phenyl-3H-1,2,4-triazoline-3,5(4H)-diones), such as that shown in FIG. 6 depicting use of a PTAD-Peg-Azide. In other embodiments, the reaction utilizes a Methionine linkage via modified oxaziridine linker, such as that shown in FIG. 5.

Biorthogonal reactions refer to chemical reactions that can take place in biological environment without affecting biomolecules or interfering with biochemical processes of that environment. Biorthogonal chemistry allows organic synthesis ordinarily performed in a laboratory to be performed in living organisms and cells. Unlike many reactions in the laboratory, however, biorthogonal reactions are not intended to prepare large amounts of material. Instead, they are intended to covalently modify biomolecules with non-native functional groups under biological conditions.

In one aspect, the biorthogonal reaction is a strain-promoted azide-alkyne click cycloaddition (SPAAC) reaction (shown below). In these reactions, cyclic alkynes are strained because bonds to the sp-hybridized alkynyl carbons normally oriented at 180° angles are pulled back because of the ring containing them. The resultant strain increases the rates of reactions that relieve the strain at the alkyne moiety.

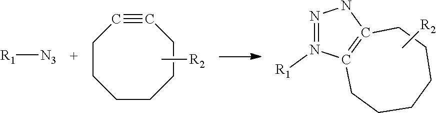

In these reactions, one reactive group is an azide and another reactive group is a cyclic alkyne. In some embodiments, the cyclic alkyne is a cyclooctyne. Cyclooctynes for use in this reaction include dibenzylcyclooctyne (DIBO), dibenzoazacyclooctyne (DBCO), and biarylazacyclooctynone (BARAC), aza-dibenzocyclooctynes (DIBAC), or a derivatives thereof. In exemplary embodiments, the cyclooctyne is a dibenzoazacyclooctyne (DBCO).

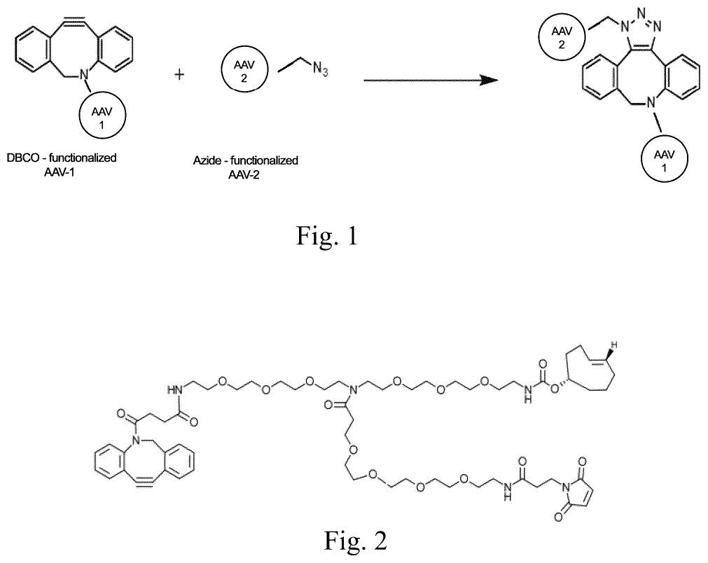

As can be seen in FIG. 1, in embodiments that utilize a SPAAC reaction, the step of conjugation a first AAV vector (shown as AAV 1 in FIG. 1) comprises conjugation with an azide moiety or a cycloctyne moiety. These reactive functional groups (henceforth referred to as a first surface moiety) are functionalized on the surface of the first or second AAV particle. The step of conjugation of a second AAV vector (shown as AAV 2 in FIG. 1) comprises conjugation with an opposite moiety than the one functionalized on the first AAV 1 particle. The second AAV 2 vector will be functionalized with either the azide or cyclooctyne (depending on which molecule was chosen for the surface functionalization of the first AAV 1 vector). Hence, in certain embodiments, the first AAV 1 vector is functionalized with a cyclooctyne (DBCO in FIG. 1) and the second AAV 2 vector is functionalized with an azide, or vice versa. The SPAAC click reaction occurs when these two vectors are combined, such that the azide and the cyclooctyne react to form a covalent linkage between the first and second AAV vectors. This physically links the two vectors, so that during delivery to a cell or to tissue, entry of one AAV vector into the cell will necessarily mean that the second AAV vector will also enter, as the two particles are now physically linked together through a covalent linkage. The covalent linkage is biorthogonal and will not interact with other molecules in the biological environment where it is delivered.

In some embodiments, the covalent linkage formed between the first surface moiety and the second surface moiety comprises the following structure 1:

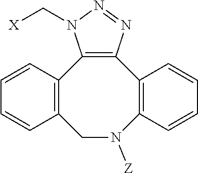

In the resulting structure 1, X can represent either a linking structure to the first AAV vector or the second AAV vector, and Z can represent either a linking structure the first AAV vector or a linking structure the second AAV vector.

As can be seen in FIG. 1, structure 1 results from a SPAAC click reaction wherein dibenzoazacyclooctyne (DBCO) is the cyclooctyne that is reacted with an azide surface moiety.

In other embodiments, the biorthogonal reaction, which creates the covalent linkage between a first AAV vector and a second AAV vector, is a copper (Cu) catalyzed azide-alkyne cycloaddition reaction (CuAAC) (shown below).

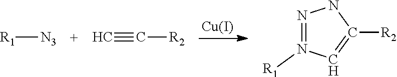

In this reaction the azide (1,3-dipole) reacts with an alkyne (dipolarophile) to produce a 1,2,3-triazole, as shown above. However, the application of CuAAC to biological systems can be a challenge due to the use of the copper catalysts; the Cu(II) precursors used in CuAAC cause oxidative damage to cells, while Cu(I) is readily oxidized to Cu(II), requiring added reductants such as ascorbate whose byproducts can also damage cells. To make CuAAC suitable for in vivo studies, extensive efforts to stabilize Cu catalysts using various ligands have been reported, most notably the use of substituted tris (triazolylmethyl) amines. The tris (triazolyl) amine ligands have demonstrated success in enhancing reaction rates and in reducing the toxicity of the copper catalyst by minimizing copper redox reactions; in addition, ligand modifications have improved the cellular permeability of copper catalysts, making them more useful in living systems.

In other embodiments, the covalent linkage is created by a strain-promoted alkyne-nitrone cycloaddition (SPANC) reaction. In this reaction, diaryl-strained-cyclooctynes, including dibenzylcyclooctyne (DIBO), can be utilized to react with 1,3-nitrones in strain-promoted alkyne-nitrone cycloaddition reaction to yield N-alkylated isoxazolines.

In further aspects, the covalent linkage between a first AAV vector and a second AAV vector is created by an alkene and tetrazine inverse electron demand Diels-Alder reaction (IEDDA). Strained cyclooctenes and other activated alkenes react with tetrazines in an inverse electron demand Diels-Alder followed by a retro [4+2] cycloaddition. In one embodiment, the first surface moiety and second surface moieties comprise a triazine, a tetrazine, or a strained dienophile, such as noroborene, transcyclooctene (TCO), cyclopropene, or N-acylazetine.

In some embodiments, the covalent linkage formed between the first surface moiety and the second surface moiety comprises the following Structure 3:

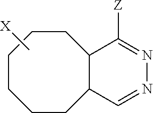

In the resulting Structure 2, X can represent either a linking structure the first AAV vector or the second AAV vector, and Z can represent a linking structure to the first AAV vector or to the second AAV vector.

The linking structure to the first AAV vector and/or second AAV vector can be a linking structure or linking molecule which is attached the first surface moiety and second surface moiety prior to their reaction with the first AAV vector and/or second AAV vector. The linking structure reacts with active groups on the surface of the AAV capsid proteins. The AAV capsid, the viruses' outer protein shell, is comprised of various functional proteins, which are comprised of amino acid sequences. In some embodiments, the linking structure can covalently bond with an amino acid residue on an AAV capsid protein, such as a free lysine amino acid residue, or terminal cysteine residues that can be expressed artificially into viral capsid protein sequences, which can be used as a linkage target. In some embodiments, viral capsid protein sequences are genetically engineered to create specific amino acid binding targets. For example, a terminal cysteine residue can be utilized by expression through genetic modifications (on the viral coat proteins of AAVs), which are then be reacted with a maleimide-peg-DBCO modifier. With this method, the number of and specific location of binding targets on a viral particle can be designed and controlled, to build a specific AAV binding mechanism with multiple binding sites on various AAVs to be linked together.

In some embodiments, the linking structure, which is attached to the first or second surface moieties, comprises a functional group that is capable of reacting with amine functional groups of a capsid protein on the outer shell of the AAVs. Amine functional groups exist at the N-terminus of each capsid protein and in the side-chain of lysine (Lys, K) amino acid residues in the capsid protein sequence. Exemplary chemical groups that react with amines include isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters.

In some embodiments, the linking structure of the first surface moiety or second surface moiety comprises N-hydroxysuccinimide ester (NHS ester) or an imidoester or PTAD. NHS esters are reactive groups formed by carbodiimide-activation of carboxylate molecules. The NHS ester-linking structure reacts with primary amines in physiologic to slightly alkaline conditions to yield stable amide bonds. The reaction releases N-hydroxysuccinimide (NHS).

In addition to the above listed chemical groups, the linking structure may also further incorporate one or more spacer structures. In some embodiments the spacer structure comprises one or more monomers of ethylene glycol, such as polyethylene glycol, or [PEG] n, also known as “dPEG n” for “discrete polyethylene glycol”, where “n” is the number of ethylene oxide (or “ethylene glycol”) units. In certain embodiments, n is 0. In certain embodiments, n is an integer between 1-25, 1-50, 1-100, 1-1000 or 1-10,000, or any value therebetween.

For example, a linking structure and spacer structure, attached a first or second surface moiety can be an NHS-PEG4 ester. Thus, the linking structure combined with the first or second surface moiety, in one embodiment, comprises an NHS-PEG4-azide ester or NHS-PEG4-DBCO ester (with either the azide or DBCO being interchangeable as the first or second surface moieties). Therefore, in this example, the step of functionalizing a first or second AAV vector comprises reacting a free lysine residue on the surface of the first or second AAV vector with an NHS-PEG4-azide ester or an NHS-PEG4-DBCO ester. Other amino acid capable of reaction with a surface moiety include at least cysteine, tyrosine, and methionine.

In one embodiment, the AAV vectors which are to be physically linked together comprise vectors from the same serotype or vectors from different serotypes. The AAV vectors can be selected from naturally occurring serotypes AAV1, AAV2, AAV3, AAV3b, AAV4, AAV5, AAV6, AAV7, AAV8, AAV9, AAV10, AAV11, AAV12, and AAV13. The serotypes are designated based on the type of surface proteins present in the capsid of the AAV particle and their specific tissue tropism. Different AAV serotypes have different binding receptors and tissue tropisms, the majority of which are already well known in the art.

In other embodiments, the AAV particles may be chosen among synthetic serotypes generated by synthetic methods, such as, but not limited to: capsid mutagenesis, peptide insertions into, or deletions from the capsid sequence, capsid shuffling from various serotypes or ancestral reconstruction.

The AAV vectors for use with the present disclosure are produced by any method known in the art. For example, the AAV vectors can be produced by various methods including: transient transfection of HEK293 cells, stable cell lines infected with Adenovirus or HSV, mammalian cells infected with Adenovirus or HSV (expressing rep-cap and transgene) or insect cells infected with baculovirus vectors (expressing rep-cap and transgene). In certain embodiments, the vectors are produced by transient transfection of HEK293 cells with calcium phosphate-HeBS method with two plasmids: pHelper, PDP2-KANA encoding AAV Rep2-Cap2 and adenovirus helper genes (E2A, VA RNA, and E4) and pVector ss-CAG-eGFP, by methods known in the art.

Although exemplary embodiments are provided with respect to adeno-associated viral vectors, the concepts disclosed herein can similarly be applied to other types of viruses, which are capable of modification to create a physical linkage for use in gene delivery therapies. For example, the physical linkages disclosed herein can be created between viral particles, including adenoviruses, retroviruses, lentiviruses, pox viruses, alphaviruses, herpes viruses and vaccinia viruses.

In addition to use of different virus particles (not limited to AAV), in some embodiments more than two viral particles are covalently linked together through, for example, a heterofunctional linkage structure. For example, 3, 4, 5, 6, 7, 8, 9, or 10 viral vectors can be physically linked through a heterofunctional linkage system having corresponding functional groups which can form covalent attachment to the various viral particles.

In one embodiment, a method of creating a linkage between three or more viral vectors is disclosed. The method comprises the steps of:
functionalizing a surface of first viral vector with a first reactive group;
functionalizing a surface of a second viral vector with a second reactive group;
functionalizing a surface of a third viral vector with a third reactive group;
providing a heterofunctional linking molecule; and
reacting the first, second and third viral vectors with the heterofunctional linking molecule. The first, second and third reactive moieties attach to the heterofunctional linking molecule to form a covalently linked structure comprising the first, second and third viral vectors.

In further embodiments, the heterofunctional linking molecule is a heterotrifunctional molecule, or a dendrimer molecule.

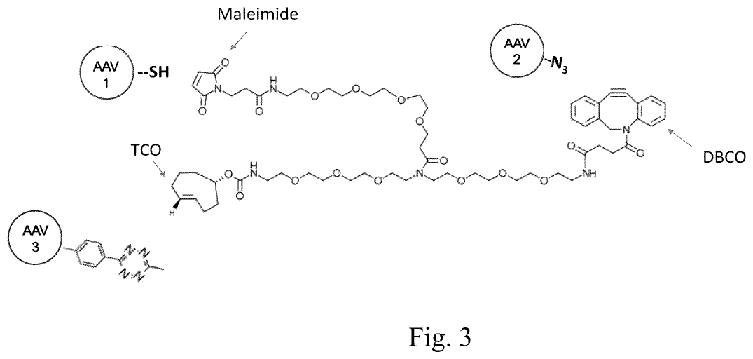

In one embodiment, three viral particles are linked together through a heterotrifunctional linker, or linking molecule. For example, a heterotrifunctional system, as shown in FIGS. 2 and 3 is disclosed, wherein a heterotrifunctional linking molecule is used to attach three different viral particles. The heterotrifunctional linking molecule has three different reactive moieties attached thereto. Each reactive moiety is capable of linking to a different viral particle, depending on a specifically designed linkage system, that includes surface functionalities on the capsid proteins of each viral particle, which will bind to the specific reactive moieties of the heterotrifunctional linking molecule.

In one embodiment, the step of reacting the first, second and third viral vectors with the heterofunctional linking molecule comprises at least one of a strain-promoted azide-alkyne click cycloaddition (SPAAC) reaction, a copper catalyzed Azide-Alkyne Cycloaddition (CuAAC) reaction, a Staudinger ligation reaction, a strain-promoted alkyne-nitrone cycloaddition (SPANC) reaction, an inverse electron demand Diels-Alder (IEDDA) reaction, or a combination thereof.

In certain embodiments, first, second or third reactive group on the surface of the functionalized first, second or third viral vectors are selected from cyclooctynes, transcyclooctenes, amine groups, sulfhydryl groups, maleimide, azide, tetrazine, triazines, phosphine, nitrone, or a combination thereof.

The reactive moieties on a heterofunctional molecule can include any of the previously discussed surface moieties, disclosed in the above embodiments. These can include cyclooctynes such as dibenzylcyclooctyne (DIBO), dibenzoazacyclooctyne (DBCO), biarylazacyclooctynone (BARAC), aza-dibenzocyclooctynes (DIBAC). Other reactive moieties include, Tetrazine, maleimide, phosphines, transcyclooctene (TCO), azide, isocyanide

For example, the heterotrifunctional linking molecule shown in FIGS. 2 and 3 has three reactive moieties: dibenzoazacyclooctyne (DBCO), transcyclooctene (TCO) and maleimide. In FIG. 3, a linkage system between three different Adenovirus particles is depicted (AAV1, AAV2 and AAV3). According to methods disclosed above, Adenovirus 1 (AAV1) is surface functionalized so as to create a reaction site for the reactive moieties of the linking molecule to bind. For example, the capsid proteins of Adenovirus 1 can be mutated to include a terminal cysteine modification, which is known to be reactive with maleimide reactive moieties. Maleimide reactive moieties can react with a surface exposed sulfhydryl group on Adenovirus 1, for example. For Adenovirus 2 (AAV2), the surface is functionalized by labelling a capsid protein with an NHS-PEG-Azide molecule, which will react via a SPAAC click reaction with a DBCO, (described in detail in previous embodiments). Adenovirus 3 (AAV3) is surface functionalized with NHS-PEG-tetrazine, which reacts with TCO reactive moieties through an inverse electron demand Diels-Alder (IEDDA) reaction. The resulting structure from these various reactions yields a linkage system having three different viral particles covalently attached together, which can be utilized as a gene delivery system.

Although the example linkage system shown in FIG. 3 discloses three different Adenovirus particles, any known type of viral particle can be utilized in this system and in other disclosed embodiments contained herein. Other viruses include: AAVs, lentiviruses, a retrovirus, herpes-simplex virus, baculo virus, vaccinia virus, or other known naturally occurring, or synthesized, genetically engineered or modified viruses.

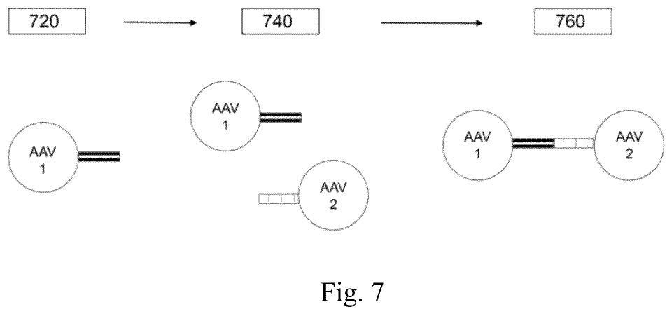

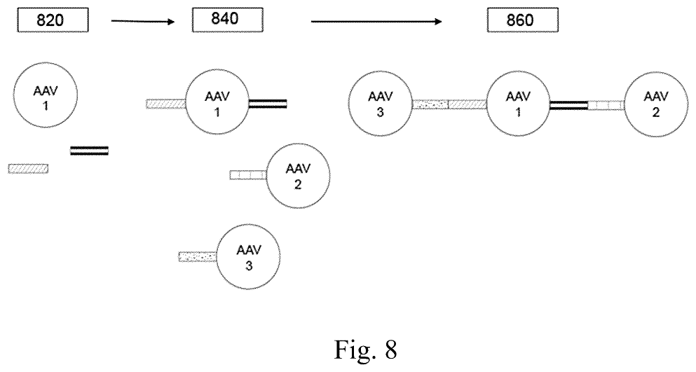

Shown in FIGS. 7, 8 and 9 are other linkage mechanism methods which are utilized for linking of multiple AAV vectors. For example, FIGS. 7 and 8 shows a linkage mechanism for two (FIG. 7) and three (FIG. 8) different AAV vectors. In the method depicted in FIG. 7 in a first step 720, AAV1 is functionalized with a first surface moiety, the functionalized AAV1 I then combined in step 740 with a second AAV vector which has been functionalized with different second surface moiety. In step 760 the first and second surface moieties react to form a covalent linkage between AAV1 and AAV2, thus resulting in a structure of two physically linked vectors. In the method depicted in FIG. 8, three AAV linked vectors are achieved. A first AAV vector is functionalized with a first surface moiety and a second surface moiety in step 820, according to any methods already described in the foregoing embodiments. In this embodiment the first and second surface moieties are different. A second functionalized AAV (AAV2) and a third functionalized AAV (AAV3) are added to the first AAV (AAV1) in step 840, to react and link the three AAV vectors in a “beads on a string” type formation, as shown in step 860 of FIG. 8. The second and third AAVs will have corresponding functionalization coupled to their capsid proteins, which can react and link with the two different surface moieties present on the surface of the first AAV (AAV1).

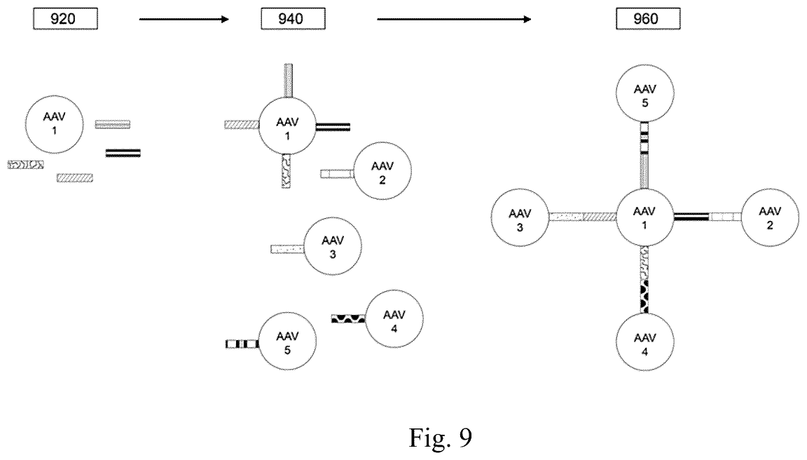

Similarly, in an additional embodiment, five AAVs are linked together through a lattice configuration, as shown in the linkage mechanism method of FIG. 9. A first AAV vector (AAV1) is functionalized with a first, second, third and fourth surface moiety shown in step 920, which can react with other surface moieties when combined with functionalized second, third, fourth and fifth AAV vectors (AAV2-AAV5), as shown in steps 940 and 960 depicted in FIG. 9. Each of the surface moieties can be chosen to selectively react only with one moiety on the surface of the first AAV vector, so as to eliminate competing reactions between AAVs and to effectively control the linkage mechanisms. In these embodiments, any linkage mechanisms, already disclosed above can be utilized, including a combination of strain-promoted azide-alkyne click cycloaddition (SPAAC) reaction, a copper catalyzed Azide-Alkyne Cycloaddition (CuAAC) reaction, a Staudinger ligation reaction, a strain-promoted alkyne-nitrone cycloaddition (SPANC) reaction, an inverse electron demand Diels-Alder (IEDDA) reaction, Tyrosine linkage via modified PTAD linkers, or Methionine linkage via modified oxaziridine linker.

The viral particles in the linked systems disclosed herein are designed to be used for purposes of delivering a genetic cargo within cells or tissue. This genetic cargo will broadly be referred to as a nucleic acid construct, for purposes of this disclosure. The nucleic acid construct packaged inside the various viral vectors of the present invention can be any kind of nucleotide sequence that is capable of transduction into cells by a recombinant virus. In further embodiments, the nucleic acid construct is capable of transcription for gene replacement, gene silencing, gene editing, gene addition or a combination thereof.

In some embodiments, the nucleic acid construct packaged inside an AAV particle is an expressible polynucleotide. In certain embodiments, the expressible polynucleotide encodes a protein. In certain embodiments, the expressible polynucleotide encodes a transgene. In certain embodiments, the expressible polynucleotide can be transcribed to provide a guide RNA, a trans-activating CRISPR RNA (tracrRNA), a messenger RNA (mRNA), a microRNA (miRNA), or a shRNA. In some embodiments, nucleic acid construct provides a DNA homology construct for homology directed repair.

In some embodiments, said nucleic acid construct is a nucleic acid molecule that is encoding extracellular antibodies (for example to neutralize certain proteins inside cells), nucleic acid molecules encoding peptide toxins (for example to block ion channels in the pain pathway), nucleic acid molecules encoding optogenetic actuators (for example to turn on or turn off neuronal activity using light), nucleic acid molecules encoding pharmacogenetic tools (for example to turn on or off neuronal signaling using chemical ligands that have no interfering pharmacological effect), nucleic acid molecules encoding CRISPR based-editors for precision gene editing, nucleic acid molecules encoding CRISPR-epigenetic tools to regulate gene expression, and/or nucleic acid molecules encoding suicide genes to induce cell death. In certain embodiments, the nucleic acid construct comprises a transgene known to be associated with a genetic disorder.

In certain embodiments, when two viral vectors are linked together, a first vector, for example a first AAV vector encapsulates a first nucleic acid construct and a second vector, and a second AAV vector, encapsulates a second nucleic acid construct. In some embodiments, the first nucleic acid construct and the second nucleic acid construct are the same. In other embodiments, the first nucleic acid construct and the second nucleic acid construct are different. This is the case for example, when different gene fragments are packaged into first AAV and second AAV, for delivery of the different genes to the same cell or tissue, for purposes of multi-gene therapies. This can also be the case where different fragments of the same large gene sequence are packaged into multiple AAV particles, for delivery to the same cell or tissue. For example, in the case of the protein dystrophin, which has a large gene sequence that cannot be packaged into a single AAV particle, but rather has to be segmented and delivered through multiple AAV vectors. Other examples include Stargardt disease (STGD1) which is caused by mutation of the ABCA4 gene, which is comprised of a 6.8 kb payload that is too large to fit inside a singular AAV with typical capacity of 4.7 kb. This disease causes macular degeneration and is a common cause of childhood onset progressive blindness. A further example is Usher disease (USH1B). One of the major forms of this disease is caused by mutation of MYO7A, whose large 7.4 kb gene is difficult to package in a single AAV.

### EXAMPLES

### Example A

Conjugation of First Viral Vector—AAV5-GFP with NHS-dPEG-Azide

According to methods described above, a first AAV vector was functionalized with NHS-dPEG4-Azide. The first AAV vector (AAV 1) was chosen to be AAV5-GFP (commercially purchased). This is a recombinant AAV serotype 5 vector which encapsulates a gene sequence for encoding a green fluorescent protein (GFP).

HEK293 cells were cultured according to known culture protocols. A 96-well plate was seeded with HEK293 cells, with 2×10⁵ cells per well. AAV-GFP vector was added to cells about 24 hours post seeding.

NHS-dPEG-Azide reagent was prepared using DMSO and diluting with PBS buffer to a concentration of 50 mg/L. 25 μL of the 50 mg/L solution was added to a microcentrifuge tube, followed by 60 μL PBS buffer. 40 μL of AAV5-GFP vector was then added to the microcentrifuge tube. The solutions were mixed and incubated for 1.5-2 hours at 4° C. Thereafter, non-reacted components were removed with a centrifugal column (3000 MWCO Microcon) by adding 500 μL PBS to a Microcon. PBS was then removed and 125 μL of the functionalized AAV5-GFP-NHS ester reaction products were added to one Microcon centrifugal column. This was centrifuged 14,000×g for 10 minutes at 4° C. AAV5-GFP-NHS-dPEG-Azide was retained and subsequently reconstituted to 125 μL with PBS.

### Example B

Conjugation of Second Viral Vector-AAV5-mCherry with NHS-dPEG-DBCO

Similarly to the method described above in Example 1, a second AAV vector was functionalized with NHS-dPEG4-DBCO. The second AAV vector (AAV 2) was chosen to be AAV5-mCherry (commercially purchased). This is a recombinant AAV serotype 5 vector which encapsulates a gene sequence for expressing mCherry, which is a monomeric red fluorescent protein.

NHS-dPEG-DBCO reagent was prepared using DMSO and diluting with PBS buffer to a concentration of 100 mg/L. 21 μL of the 100 mg/L solution was added to a microcentrifuge tube, followed by 64 μL PBS buffer. 40 μL of AAV5-mCherry vector was then added to the microcentrifuge tube. The solutions were mixed and incubated for 1.5-2 hours at 4° C. Thereafter, non-reacted components were removed with a centrifugal column (3000 MWCO Microcon) by adding 500 μL PBS to a Microcon. PBS was then removed and 125 μL of the functionalized AAV5-GFP-NHS ester reaction products were added to a Microcon centrifugal column. This was centrifuged 14,000x g for 10 minutes at 4° C. AAV5-GFP-NHS-dPEG-Azide was retained and subsequently reconstituted to 125 μL with PBS.

### Example C

### Linkage of AAV5-GFP-NHS-Azide to AAV5-m Cherry DBCO

Equal amounts of functionalized AAV5-GFP-NHS-Azide (AAV 1), obtained from Example A was added with AAV5-mCherry DBCO (AAV 2), obtained from Example B. Specifically, 100 μL of the functionalized first AAV vector (AAV 1) were combined with 100 μL of the functionalized second AAV vector (AAV2). The mixture was incubated for 1.5 to 2 hours at 4° C. while rocking. 10 μL aliquot was saved for SEM analysis.

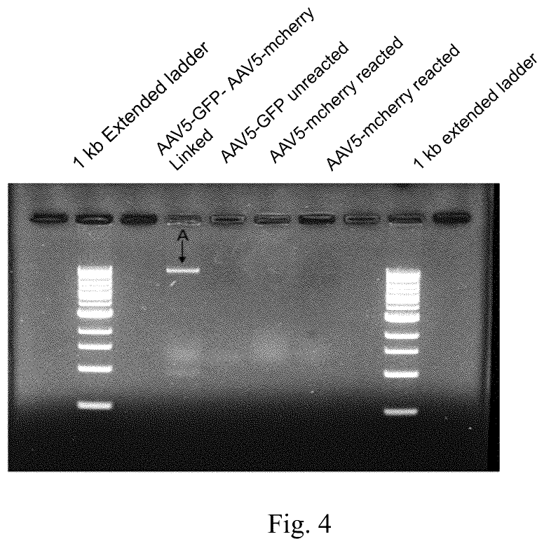

Another portion of the sample was prepared for gel-electrophoresis analysis, the result of which are shown in FIG. 4. Gel electrophoresis is a technique where DNA fragments can be separated according to their size. Well 2 and well 9 both were loaded with a 1 kb extended DNA ladder. This allows for determining the size of DNA molecules ranging from 0.5 kb (kilobases) to about 48.5 kbs. The bands at the top of the well represent larger limit of the DNA marker (i.e. larger sized DNA control), reducing in size as the ladder moves toward the bottom of the gel. The bands toward the bottom of the gel represent smaller DNA samples, with the lowest band about 250 bp. When polarized, smaller DNA samples migrate more easily down the gel than larger DNA samples do not easily move down the gel matrix due to their larger size. Hence, when the procedure is complete, the location of the bands as compared to the DNA ladders will determine a comparative size between the various samples loaded. In Well 4, the resulting linked structure from Example C was loaded, which contained the covalently linked AAV5-GFP-AAV5-mCherry. In wells 5, 6 and 7 single vectors were loaded, AAV5-GFP (unreacted) in well 5, AAV5-GFP (reacted) in well 6, and AAV5-mcherry (reacted) in well 7. The unreacted AAV vectors in well 5 do not contain any functionalized surface moieties and are single vectors. The reacted vectors in wells 6 and 7 were surface functionalized with the surface moieties, but are not linked to a second vector, and hence are still single vectors, having surface groups attached. Both the unreacted vectors show indistinct band toward the end of the ladder, whereas the linked AAV5-GFP—AAV5-mCherry in well 4, shows a strong distinct band (band A) at the top of the well. This shows that the sample loaded in the well contains a large DNA sample, particularly when compared with non-linked single AAV samples in lanes 5-7. The band (A) suggests that a successful linkage of these two AAV vectors has occurred.

The present invention is further exemplified by the following clauses: comprising:
Clause 1. A method of linking together two or more virus vectors, the method functionalizing a first virus vector with a first surface moiety;
   functionalizing a second virus vector with a second surface moiety; and
   combining the first functionalized virus vector and second functionalized virus vector;
wherein the first surface moiety and the second surface moiety react to form a covalent linkage between the first and second functionalized virus vectors.
Clause 2. The method of clause 1, wherein the first virus vector is a first Adeno-associated virus vectors (AAV) vector and the second virus vector is a second Adeno-associated virus vectors (AAV) vector, wherein the first AAV vector and/or second AAV vector are selected from a group consisting of AAV1, AAV2, AAV3, AAV3b, AAV4, AAV5, AAV6, AAV7, AAV8, AAV9, AAV10, AAV11, AAV12, AAV13 or a combination thereof.
Clause 3. The method of clause 1, wherein the reaction that forms a covalent linkage comprises a strain-promoted azide-alkyne click cycloaddition (SPAAC), a copper catalyzed Azide-Alkyne Cycloaddition (CuAAC), a Staudinger ligation reaction, a strain-promoted alkyne-nitrone cycloaddition (SPANC) reaction, an inverse electron demand Diels-Alder (IEDDA) reaction, or a combination thereof.
Clause 4. The method of clause 3, wherein the reaction that forms a covalent linkage is a strain-promoted azide-alkyne click cycloaddition reaction (SPAAC).
Clause 5. The method of clause 4, wherein the first surface moiety or the second surface moiety comprise dibenzylcyclooctyne (DIBO), dibenzoazacyclooctyne (DBCO), biarylazacyclooctynone (BARAC), aza-dibenzocyclooctynes (DIBAC), azide, or derivatives thereof.
Clause 6. The method of clause 4, wherein and the first surface moiety and/or the second surface moiety comprise dibenzocycloocytne (DBCO) or azide.
Clause 7. The method of clause 2, wherein the covalent linkage formed between the first surface moiety and the second surface moiety comprises the following structure:

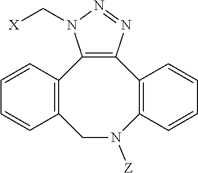

wherein X represents a linking structure of the first AAV vector or the second AAV vector, and wherein Z represents a linking structure to the first AAV vector or a linking structure to the second AAV vector.
Clause 8. The method of clause 2, wherein the first surface moiety and the second surface moiety further comprise a linking structure capable of attaching to amine groups on a capsid protein of the first AAV vector and/or the second AAV vector.
Clause 9. The method of clause 8, wherein the linking structure comprises isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters, or a combination thereof.
Clause 10. The method of clause 7, wherein linking structure X and/or linking structure Z comprise an N-hydroxysuccinimide ester (NHS) and a spacer structure comprised of one or more ethylene glycol monomers.
Clause 11. The method of clause 1, wherein the first surface moiety or the second surface moiety comprises a NHS-PEG4-azide ester or NHS-PEG4-DBCO ester.
Clause 12. The method of clause 3, wherein the reaction that forms a covalent linkage comprises is an inverse electron demand Diels-Alder (IEDDA) reaction.
Clause 13. The method of clause 12, wherein the first surface moiety or second surface moieties comprise a triazine, a tetrazine, or a strained dienophile, such as noroborene, transcyclooctene (TCO), cyclopropene, or N-acylazetine, or a combination thereof.
Clause 14. The method of clause 3, wherein the reaction that forms a covalent linkage comprises is a strain-promoted alkyne-nitrone cycloaddition (SPANC) reaction, wherein the first surface moiety or second surface moieties comprise an azide or a nitrone.
Clause 15. The method of clause 2, wherein the first AAV vector encapsulates a first nucleic acid construct and the second AAV vector encapsulates a second nucleic acid construct.
Clause 16. The method of clause 15, wherein the first nucleic acid construct and the second nucleic acid construct are the same.
Clause 17. The method of clause 15, wherein the first nucleic acid construct and the second nucleic acid construct are different.
Clause 18. The method of clause 15, wherein the first nucleic acid construct and the second nucleic acid construct are selected from a group consisting of a nucleic acid sequence, a gene fragment, a full gene sequence, a DNA fragment, an RNA fragment, mRNA, gRNA, microRNA, shRNA, CRISPR RNA, a polynucleotide, or combinations thereof.
Clause 19. The method of clause 15, wherein the first or second nucleic acid construct is capable of transcription for gene replacement, gene silencing, gene editing, or a combination thereof.
Clause 20. The method of clause 15, wherein first nucleic acid construct comprises a first gene fragment and the second nucleic acid construct comprises a second gene fragment of the same gene.
Clause 21. A covalently linked structure, according to the method of clause 2, comprising at least the first AAV vector and the second AAV vector.
Clause 22. The covalently linked structure of clause 21, wherein the structure is administered to a subject in need of therapeutic gene transfer, gene editing, or gene addition, or a combination thereof.
Clause 23. A pharmaceutical composition comprising at least one of the covalently linked structure of clause 15.
Clause 24. A composition for use in gene delivery therapies to a cell or tissue, the composition comprising at least one covalently linked structure of clause 15.
Clause 25. A method of treating a subject comprising administering to the subject a therapeutically effective amount of the pharmaceutical composition of clause 23.
Clause 26. A method of creating a linkage between three or more viral vectors, the method comprising:
   functionalizing a surface of a first viral vector with a first reactive group;
   functionalizing a surface of a second viral vector with a second reactive group;
   functionalizing a surface of a third viral vector with a third reactive group;
   providing a heterofunctional linking molecule; and
   reacting the first, second and third viral vectors with the heterofunctional linking molecule;
wherein the first, second and third reactive groups attach to the heterofunctional linking molecule to form a covalently linked structure comprising the first, second and third viral vectors.
Clause 27. The method of clause 26, wherein the first, second and third viral vectors are selected from a group consisting of adeno-associated virus, adenoviruses, retroviruses, lentiviruses and vaccinia virus, or a combination thereof.
Clause 28. The method of clause 26, wherein the first, second and third viral vectors are adeno-associate virus vectors selected from a group consisting of AAV1, AAV2, AAV3, AAV3b, AAV4, AAV5, AAV6, AAV7, AAV8, AAV9, AAV10, AAV11, AAV12, AAV13 or a combination thereof.
Clause 29. The method of clause 26, wherein the heterofunctional linking molecule is a heterotrifunctional linking molecule or a dendrimer molecule.
Clause 30. The method of clause 29, wherein the heterotrifunctional linking molecule has the following structure:

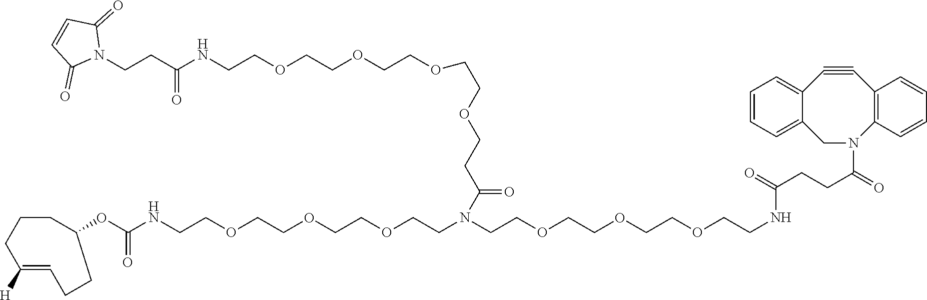

Clause 31. The method of clause 26, wherein the heterofunctional linking molecule has reactive moieties selected from cyclooctynes, transcyclooctenes, maleimide, azide, tetrazine, triazines, phosphine, nitrone, modified oxaziridine with azide functional group, PTAD, or a combination thereof.
Clause 32. The method of clause 26, wherein the first, second or third reactive group are selected from cyclooctynes, transcyclooctenes, amine groups, sulfhydryl groups, maleimide, azide, tetrazine, triazines, phosphine, nitrone, or a combination thereof.
Clause 33. The method of clause 26, wherein the step of reacting the first, second and third viral vectors with the heterofunctional linking molecule comprises at least one of a strain-promoted azide-alkyne click cycloaddition (SPAAC) reaction, a copper catalyzed Azide-Alkyne Cycloaddition (CuAAC) reaction, a Staudinger ligation reaction, a strain-promoted alkyne-nitrone cycloaddition (SPANC) reaction, an inverse electron demand Diels-Alder (IEDDA) reaction, or a combination thereof.
Clause 34. The method of clause 26, wherein reacting the first, second and third viral vectors with the heterofunctional linking molecule comprises at least one strain-promoted azide-alkyne click cycloaddition reaction (SPAAC), wherein a first, second or third reactive moiety comprises dibenzylcyclooctyne (DIBO), dibenzoazacyclooctyne (DBCO), biarylazacyclooctynone (BARAC), aza-dibenzocyclooctynes (DIBAC), azide, or derivatives thereof.
Clause 35. The method of clause 26, wherein the first, second or third reactive groups further comprise an N-hydroxysuccinimide ester (NHS) and a spacer structure comprised of one or more ethylene glycol monomers.
Clause 36. The method of clause 26, wherein the first, second or third reactive groups comprises a NHS-PEG4-azide ester, a sulfhydryl group, or NHS-PEG4-DBCO ester.
Clause 37. The method of clause 26, wherein the first viral vector encapsulates a first nucleic acid construct, the second viral vector encapsulates a second nucleic acid construct, and the third viral vector encapsulates a third nucleic acid construct.
Clause 38. The method of clause 26, wherein the first nucleic acid construct, second nucleic acid construct and third nucleic acid construct are the same.
Clause 39. The method of clause 26, wherein the first nucleic acid construct, the second nucleic acid construct, and/or the third nucleic acid construct are different.
Clause 40. The method of clause 37, wherein the first nucleic acid construct, the second nucleic acid construct or third nucleic acid construct are selected from a group consisting of a nucleic acid sequence, a gene fragment, a full gene sequence, a DNA fragment, an RNA fragment, mRNA, gRNA, microRNA, shRNA, CRISPR RNA, a polynucleotide, or combinations thereof.
Clause 41. The method of clause 37, wherein the first, second or third nucleic acid construct is capable of transcription for gene replacement, gene silencing, gene editing, or a combination thereof.
Clause 42. The method of clause 37, wherein first nucleic acid construct comprises a first gene fragment, the second nucleic acid construct comprises a second gene fragment, and the third nucleic acid construct comprises a third gene fragment, wherein the first, second and third gene fragments are from the same gene.
Clause 43. A covalently linked structure, according to the method of clause 37, comprising at least a first viral vector, a second viral vector, and a third viral vector.
Clause 44. The covalently linked structure of clause 37, wherein the structure is administered to a subject in need of therapeutic gene transfer, gene editing, or gene addition, or a combination thereof.
Clause 45. A pharmaceutical composition comprising at least one of the covalently linked structure of clause 43.
Clause 46. A composition for use in gene delivery therapies to a cell or tissue, the composition comprising at least one covalently linked structure of clause 43.
Clause 47. A method of treating a subject comprising administering to the subject a therapeutically effective amount of the pharmaceutical composition of clause 44.

While exemplary embodiments are described above, it is not intended that these embodiments describe all possible forms encompassed by the clauses. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, case of assembly, etc. As such, to the extent any embodiments are described as less desirable than other embodiments or prior art implementations with respect to one or more characteristics, these embodiments are not outside the scope of the disclosure and can be desirable for particular applications.

1. A method of linking together two or more virus vectors, the method comprising:
functionalizing a first virus vector with a first surface moiety;
functionalizing a second virus vector with a second surface moiety; and
combining the first functionalized virus vector and second functionalized virus vector;
wherein the first surface moiety and the second surface moiety react to form a covalent linkage between the first and second functionalized virus vectors.

2. The method of claim 1, wherein the first virus vector is a first Adeno-associated virus vectors (AAV) vector and the second virus vector is a second Adeno-associated virus vectors (AAV) vector, wherein the first AAV vector and/or second AAV vector are selected from a group consisting of AAV1, AAV2, AAV3, AAV3b, AAV4, AAV5, AAV6, AAV7, AAV8, AAV9, AAV10, AAV11, AAV12, AAV13 or a combination thereof.

3. The method of claim 1, wherein the reaction that forms a covalent linkage comprises a strain-promoted azide-alkyne click cycloaddition (SPAAC), a copper catalyzed Azide-Alkyne Cycloaddition (CuAAC), a Staudinger ligation reaction, a strain-promoted alkyne-nitrone cycloaddition (SPANC) reaction, an inverse electron demand Diels-Alder (IEDDA) reaction, or a combination thereof.

4. The method of claim 3, wherein the reaction that forms a covalent linkage is a strain-promoted azide—alkyne click cycloaddition reaction (SPAAC).

5. The method of clause 4, wherein the first surface moiety or the second surface moiety comprise dibenzylcyclooctyne (DIBO), dibenzoazacyclooctyne (DBCO), biarylazacyclooctynone (BARAC), aza-dibenzocyclooctynes (DIBAC), azide, or derivatives thereof.

6. The method of claim 4, wherein and the first surface moiety and/or the second surface moiety comprise dibenzocycloocytne (DBCO) or azide.

7. The method of claim 2, wherein the covalent linkage formed between the first surface moiety and the second surface moiety comprises the following structure: 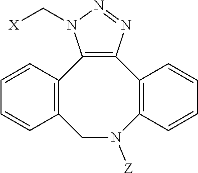
wherein X represents a linking structure of the first AAV vector or the second AAV vector, and wherein Z represents a linking structure to the first AAV vector or a linking structure to the second AAV vector.

8. The method of claim 2, wherein the first surface moiety and the second surface moiety further comprise a linking structure capable of attaching to amine groups on a capsid protein of the first AAV vector and/or the second AAV vector.

9. The method of claim 8, wherein the linking structure comprises isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters, or a combination thereof.

10. The method of claim 7, wherein linking structure X and/or linking structure Z comprise an N-hydroxysuccinimide ester (NHS) and a spacer structure comprised of one or more ethylene glycol monomers.

11. The method of claim 1, wherein the first surface moiety or the second surface moiety comprises a NHS-PEG4-azide ester or NHS-PEG4-DBCO ester.

12. The method of claim 3, wherein the reaction that forms a covalent linkage comprises is an inverse electron demand Diels-Alder (IEDDA) reaction.

13. The method of claim 12, wherein the first surface moiety or second surface moieties comprise a triazine, a tetrazine, or a strained dienophile, such as noroborene, transcyclooctene (TCO), cyclopropene, or N-acylazetine, or a combination thereof.

14. The method of claim 3, wherein the reaction that forms a covalent linkage comprises is a strain-promoted alkyne-nitrone cycloaddition (SPANC) reaction, wherein the first surface moiety or second surface moieties comprise an azide or a nitrone.

15. The method of claim 2, wherein the first AAV vector encapsulates a first nucleic acid construct and the second AAV vector encapsulates a second nucleic acid construct.

16. The method of claim 15, wherein the first nucleic acid construct and the second nucleic acid construct are the same.

17. The method of claim 15, wherein the first nucleic acid construct and the second nucleic acid construct are different.

18. The method of claim 15, wherein the first nucleic acid construct and the second nucleic acid construct are selected from a group consisting of a nucleic acid sequence, a gene fragment, a full gene sequence, a DNA fragment, an RNA fragment, mRNA, gRNA, microRNA, shRNA, CRISPR RNA, a polynucleotide, or combinations thereof.

19. The method of claim 15, wherein the first or second nucleic acid construct is capable of transcription for gene replacement, gene silencing, gene editing, or a combination thereof.

20. The method of claim 15, wherein first nucleic acid construct comprises a first gene fragment and the second nucleic acid construct comprises a second gene fragment of the same gene.

21. A covalently linked structure, according to the method of claim 2, comprising at least the first AAV vector and the second AAV vector.

22. The covalently linked structure of claim 21, wherein the structure is administered to a subject in need of therapeutic gene transfer, gene editing, or gene addition, or a combination thereof.

23. A method of creating a linkage between three or more viral vectors, the method comprising:
functionalizing a surface of a first viral vector with a first reactive group;
functionalizing a surface of a second viral vector with a second reactive group;
functionalizing a surface of a third viral vector with a third reactive group;
providing a heterofunctional linking molecule; and
reacting the first, second and third viral vectors with the heterofunctional linking molecule;
wherein the first, second and third reactive groups attach to the heterofunctional linking molecule to form a covalently linked structure comprising the first, second and third viral vectors.

24. The method of claim 23, wherein the first, second and third viral vectors are selected from a group consisting of adeno-associated virus, adenoviruses, retroviruses, lentiviruses and vaccinia virus, or a combination thereof.

25. The method of claim 23, wherein the first, second and third viral vectors are adeno-associate virus vectors selected from a group consisting of AAV1, AAV2, AAV3, AAV3b, AAV4, AAV5, AAV6, AAV7, AAV8, AAV9, AAV10, AAV11, AAV12, AAV13 or a combination thereof.

26. The method of claim 23, wherein the heterofunctional linking molecule is a heterotrifunctional linking molecule or a dendrimer molecule.

27. The method of claim 26, wherein the heterotrifunctional linking molecule has the following structure:

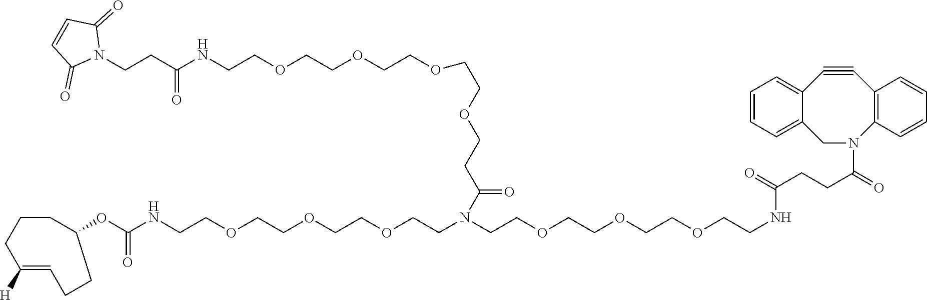

28. The method of claim 23, wherein the heterofunctional linking molecule has reactive moieties selected from cyclooctynes, transcyclooctenes, maleimide, azide, tetrazine, triazines, phosphine, nitrone, modified oxaziridine with azide functional group, PTAD, or a combination thereof.

29. The method of claim 23, wherein the first, second or third reactive group are selected from cyclooctynes, transcyclooctenes, amine groups, sulfhydryl groups, maleimide, azide, tetrazine, triazines, phosphine, nitrone, or a combination thereof.

30. The method of claim 23, wherein the step of reacting the first, second and third viral vectors with the heterofunctional linking molecule comprises at least one of a strain-promoted azide—alkyne click cycloaddition (SPAAC) reaction, a copper catalyzed Azide-Alkyne Cycloaddition (CuAAC) reaction, a Staudinger ligation reaction, a strain-promoted alkyne-nitrone cycloaddition (SPANC) reaction, an inverse electron demand Diels-Alder (IEDDA) reaction, or a combination thereof.
